(19) United States
(12) Patent Application Publication
(10) Pub. No.: US 2011/0269846 A1
(43) Pub. Date: Nov. 3, 2011

(54) Formulations with high percentage cis-p-menthan-7-ol and preparation thereof

(75) Inventors: Robert H. Bedoukian, West Redding, CT (US); Manfred Pawlak, Princeton, NJ (US)

(73) Assignee: Bedoukian Research, Inc.

(21) Appl. No.: 12/799,682

(22) Filed: Apr. 29, 2010

(51) Int. Cl. A61K 8/34; A01P 7/04; A61Q 13/00; C11D 3/50; A01N 25/00

(52) U.S. Cl. 514/772; 512/25; 510/101

(57) ABSTRACT

Perfumed compositions or articles, and particularly perfumes, soaps, cosmetics, aftershaves, eye creams, insecticides and antiseptics formulations, containing p-menthane-7-ol wherein the cis portion of the p-menthane-7-ol exceeds 90% by weight, preferably comprises 93% or more, and more preferably comprises 95% or more, provide greatly improved fragrance characteristics wherein the odor produced is a more natural, strong, fresh muguet lily-of-the valley fragrance.

### FIELD OF THE INVENTION

This invention relates to preparation of high percentage cis-p-menthan-7-ol, and use of such high percentage cis-p-menthan-7-ol as a perfuming ingredient for the preparation of perfuming compositions and perfumed articles to which a high percentage cis-p-menthan-7-ol imparts a uniquely strong and fresh muguet note.

### BACKGROUND TO THE INVENTION

Already in 1908 it was discovered that the compound 3,7-dimethyl-7-hydroxyoctanal or hydroxycitronellal has a beautiful lily-of-the-valley-like (French-muguet) odor. This famous muguet odorant is used in countless perfumes for this nuance. It is used in the creation and/or manufacturing of fragrance and flavor concentrates of all types. Hydroxycitronellal has a sweet, floral odor and therefore is often used in perfumes, soaps, cosmetics, aftershaves, eye creams, insecticides and antiseptics. Unfortunately it is rather unstable and has shown to be slightly allergenic and known to be a human immune system toxicant, so its use level is restricted. A hydroxycitronellal allergy is an adverse reaction by the body's immune system to the chemical called hydroxycitronellal found in perfumes or fragrances. The body's immune system produces immunoglobulin E (IgE—an antibody) and histamine in response to contact with the allergen. The specific symptoms that can result can vary considerably amongst patients from a severe anaphylactic reaction to asthma, abdominal symptoms, eczema or headaches. Although it has some disadvantages (weak odor, stability problems, irritating effect on skin), it's the best aroma chemical reproducing the lily-of-the valley odor. The problem is that because of its rather weak smell it needs to be used in relatively high amounts to make a noticeable effect. But from another side it irritates the skin at high concentrations. IFRA has restricted the use of hydroxycitronellal to a maximum of 1% in perfumes that makes problematic the use of high concentrations of this aroma chemical in fragrances at perfume concentration. But it is still enough to make a decent Muguet soliflore at EdT or even EdP concentration. In view of these problems with hydroxycitronellal and the continuing desire to be able to produce a lily-of-the valley like odor other compounds have been sought.

In view of the desire to be able to continue to produce an acceptable muguet odor or fragrance there have been attempts to develop alternative chemicals to produce such results without having such undesirable effects as those obtained with hydroxycitronellal. In that regard a number of stable alcohols have been developed as alternatives to hydroxycitronellal. Among the most widely accepted and used alcohol for this purpose of providing an alternative muguet odor or fragrance is p-menthan-7-ol sold commercially as Mayol™ and as Meijiff™. However, although the commercially available Mayol™ and Meijiff™ p-menthan-7-ol, is sold as cis-p-menthan-7-ol, they are products that contain only 70% cis isomer and 30% trans isomer, and it is these products that has been used as a fragrance or odor producer in perfumes, soaps, cosmetics, aftershaves, eye creams, insecticides and antiseptics. These products, while producing a somewhat muguet fragrance, are certainly not equivalent to the sweet floral fragrance provided by hydroxycitronellal.

Thus, there is a need for an aroma chemical that can match or exceed the muguet floral fragrance heretofore provided by hydroxycitronellal without the undesirable side effect experienced from the use of hydroxycitronellal as a fragrance.

### SUMMARY OF THE INVENTION

We have discovered that the presence of a high percentage (10% or more and especially 30% as found in the commercially available products) of trans-p-menthan-7-ol in a -p-menthan-7-ol composition imparts a powerful cumin-like odor or fragrance and causes the p-menthan-7-ol composition to have diminished muguet floral effect. We have further discovered that when the cis isomer exceeds 90%, preferably comprises 93% or more, and more preferably comprises 95% or more, of a p-menthan-7-ol composition the odor or fragrance of the p-menthane-7-ol composition changes dramatically and becomes a very clean, bright floral/muguet note. Thus, we have discovered that perfumed compositions and articles containing p-menthane-7-ol wherein the cis portion of the p-menthane-7-ol exceeds 90%, preferably exceeds 93% and more preferably exceeds 95%, provides greatly improved fragrance characteristics wherein the aroma produced is a more natural, strong, fresh muguet lily-of-the valley fragrance. The p-menthane-7-ol wherein the cis portion of the p-menthane-7-ol exceeds 90%, preferably exceeds 93% and more preferably exceeds 95%, can de incorporated into any composition, formulation or article wherein it is desired to obtain the muguet fragrance or aroma. Examples of such compositions, formulations and articles include soap, a cosmetic, an aftershave, an eye cream, an insecticide or an antiseptic formulation, shampoos, detergent products, air fresheners and the like. Incorporation of such p-menthane-7-ol wherein the cis portion of the p-menthane-7-ol exceeds 90%, preferably exceeds 93% and more preferably exceeds 95%, into soap, a cosmetic, an aftershave, an eye cream, an insecticide or an antiseptic formulation is especially preferred.

### DETAILED DESCRIPTION OF THE INVENTION

Perfumed compositions and articles, and especially perfumes, soaps, cosmetics, aftershaves, eye creams, insecticides and antiseptics formulations containing p-menthane-7-ol wherein the cis portion of the p-menthane-7-ol exceeds 90%, preferably exceeds 93% and more preferably exceeds 95%, provides greatly improved fragrance characteristics wherein the odor produced is more naturally and strongly like natural muguet lily-of-the valley fragrance.

p-Menthane-7-ol can be prepared by the process disclosed in BASF Aktiengesellschaft's U.S. Pat. No. 4,847,425 and results in a p-menthane-7-ol containing about 30-40% trans isomer by weight. In order to obtain the p-menthane-7-ol containing in excess of 90% or more, preferably 93% or more, and more preferably 95% or more by weight of cis-isomer a 70/30 cis/trans mixture of p-menthane-7-ol was fractionally distilled. 200 Grams of 70/30 cis/trans p-menthane-7-ol was charged into a vacuum distillation unit equipped with a 3 foot tall Goodloe structured packed column and fractionating reflux head. The 70/30 mixture was distilled at 45 mm Hg with a pot temperature of 152° C. The trans p-menthane-7-ol was slowly collected away from the cis isomer at a head temperature of 145° C. and afforded 100 grams of the 97% by weight cis isomer material.

p-Menthane-7-ol wherein the cis isomer exceeds 90%, preferably exceeds 93% and more preferably exceeds 95% by weight, can be employed in any perfume, soap, cosmetic, aftershave, eye cream, insecticide or antiseptic formulations where it is desired that such formulation present a muguet floral fragrance characteristic and especially where it is desired that the odor produced is more naturally and strongly like natural muguet hydroxycitronellal lily-of-the valley fragrance.

The following are non-limiting examples of such formulations containing p-menthane-7-ol, in which the cis isomer is 93% by weight or more of the p-menthane-7-ol.

GREEN FLORAL MUGUET FRAGRANCE IN A COSMETIC

| Ingredient | Parts by Weight |
|---|---|
| Bergamote Oil Italy | 60 |
| Citronellal 10% | 3 |
| Citronellol | 1.3 |
| Dimethyl Benzyl Carbinyl Acetate | 14 |
| Dimethyl Phenyl Ethyl Carbinol | 92 |
| Dipropylene glycol | 3.4 |
| Geraniol | 1.3 |
| Cis-3-hexenol 10% | 192 |
| 3-(4-tert-butylphenyl)-2-methylpropanol | 19 |
| Linalool | 3 |
| Linalyl Acetate | 204 |
| Methyl Octine Carbonate | 11 |
| Mimosa Abs 10% | 13 |
| Nerolidol | 27 |
| Orange Oil | 5 |
| Para-Cresyl Methyl Ether 1% | 13 |
| Phenyl Acetaldehyde 10% | 6 |
| Phenyl Acetaldehyde dimethyl acetal | 31 |
| Phenyl Ethyl Alcohol | 179 |
| Syringaldehyde 50% | 69 |
| p-Menthane-7-ol >93% cis | 53 |
| Total | 1000 |

FRESH AROMATIC MUGUET FRAGRANCE IN AN AFTERSHAVE

| Ingredient | Parts by Weight |
|---|---|
| Allyl Amyl Glycolate | 1.2 |
| Ambrettolide | 0.1 |
| Benzyl Acetate | 3.3 |
| Cardamon Oil | 14 |
| Carvone Laevo 10% | 0.3 |
| Dodecahydro-6.6.9a-trimethyl- | 10 |
| naphto[2,1-b]furan | |
| Lovage Oil | 0.2 |
| Cistus Oil | 0.2 |
| Citronellol | 6 |
| Citronellyl Acetate 10% | 0.8 |
| Coumarin | 3.8 |
| Dihydromyrcenol | 64 |
| Dimethyl Anthranilate | 0.1 |
| Dipropylene glycol | 107.1 |
| Ethyl Acetoacetate | 0.1 |
| Ethyl Linalool | 11 |
| 1-(5,6,7,8-Tetrahydro-3,5,5,6,6,8,8- | 40 |
| hexamethyl-2-naphthalenyl)ethan-1-one | |
| 3-Ethylphenyl,2,2,-dimethyl- | 3.9 |
| propionaldehyde | |
| 1,3,4,6,7,8-Hexahydro-4,6,6,7,8,8- | 110 |
| hexaamethylcyclopenta-gamma- | |
| 2-benzopyran in isopropyl | |
| myristate | |
| Geraniol | 4 |
| Geranyl Acetate | 1.4 |
| Guaiacwood Oil | 5 |
| Methyl dihydrojasmonate | 150 |
| Cis-3-Hexenol | 0.2 |
| Cis-3-Hexenylacetate | 0.1 |
| Cis-3-Hexenylsalicylate | 10 |
| Ionone Beta | 26 |
| Prop-2-enyl 2-cyclohexyloxyacetate | 3 |
| 1-(1,2,3,4,5,6,7,8-octahydro-2,3,8,8- | 45 |
| tetramethyl-2-naphthalenyl)- | |
| ethan-1-one | |
| Jasmin Abs Egypt | 1 |
| Cis-Jasmone | 0.5 |
| Lemon Oil | 76 |
| Lemongrass Oil | 0.8 |
| Cis-3-hexen-1-yl methylcarbonate | 0.1 |
| 3-(4-tert-Butylphenyl)-2-methylpropanol | 13 |
| Linalool | 95 |
| Linalyl Acetate | 114 |
| Terpineol | 1 |
| Neryl Acetate | 1.5 |
| Norsandyl 81157 Firmenich | 5 |
| Nutmeg Oil | 5 |
| Phenyl Ethyl Alcohol | 18 |
| Terpinyl Acetate | 1 |
| 2,4-dimethyl-3-cyclohexene-1- | 0.8 |
| carboxaldehyde 10% | |
| Vanillin 10% | 0.5 |
| p-Menthane-7-ol >93% cis | 46 |
| Total | 982.1 |

CLEAN FRESH MUGUET FRAGRANCE IN ANTISEPTIC OR
INSECTICIDE

| Ingredient | Parts by Weight |
|---|---|
| Aldehyde C-9 10% | 5 |
| Aldehyde C-10 10% | 5 |
| Aldehyde C-12 1% | 25 |
| Cyclamen Aldehyde 1% | 5 |
| Dipropylene glycol | 769 |
| Hydroxicitronellal | 7 |
| Orange Terpenes | 4 |
| Undecalactone Gamma 1% | 10 |
| p-Menthane-7-ol >93% cis | 170 |
| Total | 1000 |

FRESH CREAMY MUGUET FRAGRANCE IN SOAP

| Ingredient | Parts by Weight |
|---|---|
| Benzyl Acetate | 35 |
| Linalyl Acetate | 45 |
| Phenyl Ethyl Acetate | 2.5 |
| Styrallyl Acetate | 2 |
| Terpinyl Acetate | 15 |
| Benzyl Alcohol | 15 |
| Aldehyde C-12 50% in isopropyl myristate | 10 |
| Hexyl cinnamic aldehyde | 15 |
| Citronellol | 170 |
| Isopropyl myristate | 4 |
| Coumarine | 80 |
| Acetic acid, p-tert-butylcyclohexyl ester | 100 |
| Eugenol | 1.5 |
| 1,3,4,6,7,8-Hexahydro-4,6,6,7,8,8- | 15 |
| hexamethyl cyclopenta (gamma)- | |
| benzopyran 50 in isopropyl | |
| myristate | |
| Geraniol | 95 |
| Indol 10% | 3.5 |
| 3-(4-tert-Butylphenyl)-2-methylpropanol | 40 |
| Linalool | 90 |
| 4-(4-Hydroxy-4-methylpentyl) 3- | 40 |
| cyclohexen-1-carboxaldehyde, | |
| Menthone | 1.5 |
| Rosinol | 15 |
| Phenyl Ethyl Alcohol | 240 |
| Terpineol | 85 |
| Tetrahydromuguol | 85 |
| Tonalide | 75 |
| p-Menthane-7-ol >93% cis | 20 |
| Total | 1300 |

MUGUET FRAGRANCE IN A PERFUME

| Ingredient | Parts by Weight |
|---|---|
| Bergamonte oil NS | 20 |
| Cardamon oil 10% | 10 |
| Methyl dihydrojasmonate | 120 |
| Damascenone 10% | 20 |
| p-Menthane-7-ol >93% cis | 66 |
| Cis-hexanol-3 10% | 2 |
| Cis-jasmone 10% | 65 |
| Linalool | 26 |
| Dipropylene glycol | 1 |
| Total | 330 |

A corresponding perfume formulation of the same ingredients, except that 66 parts by weight of p-menthane-7-ol with a 70/30 cis/trans ratio in place of Applicant's 66 parts p-menthane-7-ol >93% cis, was also prepared. The perfume of this invention with the p-menthane-7-ol >93% cis had a clean fresh citrus note while the 70/30 cis/trans ratio p-menthane-7-ol containing perfume had a slightly dirty note.

MUGUET FRAGRANCE IN A PERFUME

| Ingredient | Parts by Weight |
|---|---|
| Anisic aldehyde 10% | 10 |
| Benzyl salicylate | 300 |
| Citral extra | 2 |
| Delta Damascone 10% | 5 |
| Dihydromyrcenol | 20 |
| Dipropyleneglycol | 68 |
| Eucalyptol | 5 |
| Geraniol | 80 |
| Hexyl Salicylate | 100 |
| Hexyl cinnamic aldehyde | 200 |
| Cis-3-hexen-1-yl methyl carbonate 10% | 5 |
| 3-(4-tert-Butylphenyl)-2-methylpropanol | 50 |
| Linalool | 50 |
| p-Menthane-7-ol >93% cis | 50 |
| Methyl Benzoate | 5 |
| Neroli Oil Bigarade | 5 |
| Phenyl Ethyl Alcohol | 60 |
| Terpineol Alpha | 20 |
| Undecavertol 10% | 10 |
| Vanillin 10% | 5 |
| Verdox | 50 |
| Total | 1100 |

A corresponding perfume of the same ingredient, except that 50 parts by weight p-menthane-7-ol with a 70/30 cis/trans ratio, in place of Applicant's p-menthane-7-ol >93% cis, was also prepared. The perfume of this invention with the p-menthane-7-ol >93% cis had a cleaner and softer fresh lily of the valley citrus note than the p-menthane-7-ol with a 70/30 cis/trans ratio containing perfume had.

Perfumes containing the p-menthane-7-ol >93% cis of this invention and corresponding perfumes with p-menthane-7-ol having a 70/30 cis/trans ratio in place of this invention's p-menthane-7-ol >93% cis were prepared and given to recognized experts in the perfume industry for their evaluation of the fragrances. Their evaluations were as flows.

An independent consultant with over 46 years experience in the perfume industry commented as follows. “With an increasing concentration of the cis isomer, the odor is changing from a soft white floral, muguet note to a more powerful green, natural lily of the valley odor of great strength and tenacity. The cut off line is in my opinion at 93% cis. The fresh natural muguet character is best at 98% cis. The odor improvement at 93% cis is quite remarkable.”

A professional independent consultant with over 35 years experience in fragrance R&D, aroma chemicals research, delivery systems, fragrance applications, consumer product formulation and product safety and regulatory affairs commented as follows. “I too thought that the cis-isomer was not only elegantly floral but was surprisingly stronger. These attributes were not obvious and could form the basis of an invention.”

A professional perfumer, a graduate of the prestigious Givaudan Perfumery School, Grasse, France, and having fourteen years experiencing in designing many of today's most recognized fine fragrances commented as follows. “We finally reviewed the comparative samples I had put up last week and the good news for you is that we all agreed on the superiority of your product. At the same level in the formula (3.5%), compared to good old Mayol, p-menthane-7-ol >93% cis brings more lift, more clarity, more floral volume and a much more refined floral feel so from an olfactive stand point it is far better.”

While the invention has been described herein with reference to the specific embodiments thereof, it will be appreciated that changes, modification and variations can be made without departing from the spirit and scope of the inventive concept disclosed herein. Accordingly, it is intended to embrace all such changes, modification and variations that fall with the spirit and scope of the appended claims.

1. A perfumed composition or article containing p-menthane-7-ol wherein the cis portion of the p-menthane-7-ol exceeds 90% by weight.

2. A perfumed composition or article according to claim 1 wherein the perfumed composition is selected from the group consisting of a soap, a cosmetic, an aftershave, an eye cream, an insecticide or an antiseptic formulation.

3. A perfume, soap, cosmetic, aftershave, eye cream, insecticide or antiseptic formulation according to claim 2 wherein the cis portion of the p-menthane-7-ol comprises 93% or more by weight.

4. A perfume, soap, cosmetic, aftershave, eye cream, insecticide or antiseptic formulation according to claim 2 wherein the cis portion of the p-menthane-7-ol comprises 95% or more by weight.

5. A perfume according to claim 2.

6. A soap according to claim 2.

7. An aftershave according to claim 2.

8. An eye cream according to claim 2.

9. An insecticide according to claim 2.

10. An antiseptic according to claim 2.

11. A method for preparing a perfumed composition or article having a natural, fresh muguet lily-of-the valley fragrance comprising incorporating into such composition p-menthane-7-ol wherein the cis portion of the p-menthane-7-ol exceeds 90% by weight.

12. A method according to claim 11 wherein the perfumed composition is selected from the group consisting of a soap, a cosmetic, an aftershave, an eye cream, an insecticide or an antiseptic formulation.

13. A method according to claim 11 wherein the cis portion of the p-menthane-7-ol comprises 93% or more by weight.

14. A method according to claim 11 wherein the cis portion of the p-menthane-7-ol comprises 95% or more by weight.

15. A method according to claim 12 wherein the formulation is a perfume.

16. A method according to claim 12 wherein the formulation is a soap.

17. A method according to claim 12 wherein the formulation is an aftershave.

18. A method according to claim 12 wherein the formulation is a An eye cream.

19. A method according to claim 12 wherein the formulation is an insecticide.

20. A method according to claim 12 wherein the formulation is an antiseptic.
